(19) United States
(12) Patent Application Publication
(10) Pub. No.: US 2008/0194907 A1
(43) Pub. Date: Aug. 14, 2008

(54) Massaging Device for Sexual Purpose

(76) Inventors: Hsi-Fu Lin, Taichung City (TW); Hsu-Yi Wang, Taichung City (TW)

Correspondence Address: Dr. BANGER SHIA, 102 Lindencrest Ct., Sugar Land, TX, 77479-5201, US

(21) Appl. No.: 11/672,864

(22) Filed: Feb. 8, 2007

(51) Int. Cl. A61F 5/00; A61H 1/00

(52) U.S. Cl. 600/38; 601/70; 601/72; 601/46

(57) ABSTRACT

A massaging device for a sexual purpose includes main body, a sliding unit movably mounted on the main body linearly and reciprocally, a reciprocating mechanism mounted in the main body and connected to the sliding unit to drive the sliding unit reciprocally, and a power source mounted in the main body and connected to the reciprocating mechanism to drive the reciprocating mechanism. Thus, the sliding unit is movable on the housing of the main body linearly and reciprocally to massage a human sexual organ automatically and to provide a comfortable sensation to the human sexual organ so as to satisfy the sexual requirement.

### BACKGROUND OF THE INVENTION

1. Field of the Invention

The present invention relates to a massaging device, and more particularly to a massaging device for a sexual purpose.

2. Description of the Related Art

A conventional massaging device for a sexual purpose comprises a main body, and a motor mounted in the main body to provide a vibrating function so that the main body is vibrated automatically to rub and massage a human sexual organ, such as the female vagina or the male penis, to provide a comfortable sensation to the human sexual organ so as to satisfy the sexual requirement.

### SUMMARY OF THE INVENTION

The primary objective of the present invention is to provide a massaging device that is available for a sexual purpose to achieve an exciting effect.

Another objective of the present invention is to provide a massaging device, wherein the sliding unit is movable on the housing of the main body linearly and reciprocally to massage a human sexual organ automatically and to provide a comfortable sensation to the human sexual organ so as to satisfy the sexual requirement.

In accordance with the present invention, there is provided a massaging device, comprising a main body, a sliding unit movably mounted on the main body linearly and reciprocally, a reciprocating mechanism mounted in the main body and connected to the sliding unit to drive the sliding unit reciprocally, and a power source mounted in the main body and connected to the reciprocating mechanism to drive the reciprocating mechanism.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a massaging device in accordance with the preferred embodiment of the present invention;

FIG. 2 is an exploded perspective view of the massaging device as shown in FIG. 1;

FIG. 3 is a plan cross-sectional view of the massaging device as shown in FIG. 1;

FIG. 4 is a plan cross-sectional view of the massaging device taken along line 4-4 as shown in FIG. 3;

FIG. 5 is a plan cross-sectional view of a massaging device in accordance with another preferred embodiment of the present invention;

FIG. 6 is an exploded perspective view of a massaging device in accordance with another preferred embodiment of the present invention;

FIG. 7 is a plan cross-sectional assembly view of the massaging device as shown in FIG. 6;

FIG. 8 is a plan cross-sectional view of the massaging device taken along line 8-8 as shown in FIG. 7;

FIG. 9 is a plan cross-sectional view of a massaging device in accordance with another preferred embodiment of the present invention;

FIG. 10 is a plan cross-sectional view of a massaging device in accordance with another preferred embodiment of the present invention;

FIG. 11 is a perspective view of a massaging device in accordance with another preferred embodiment of the present invention; and

FIG. 12 is a plan cross-sectional view of the massaging device as shown in FIG. 11.

### DETAILED DESCRIPTION OF THE INVENTION

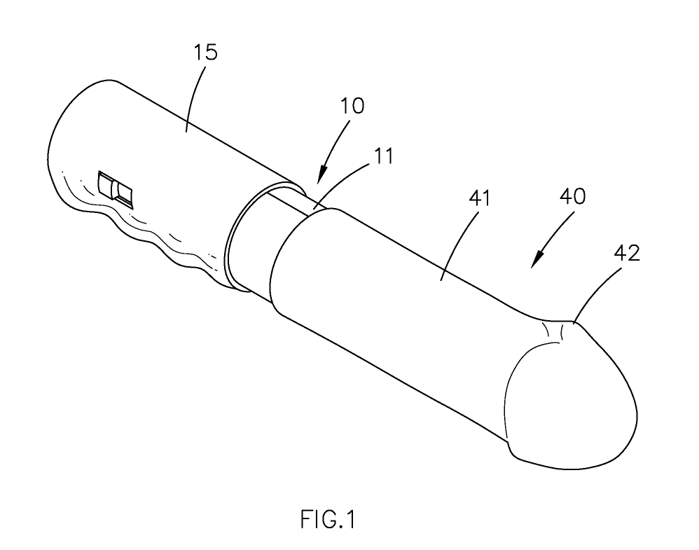

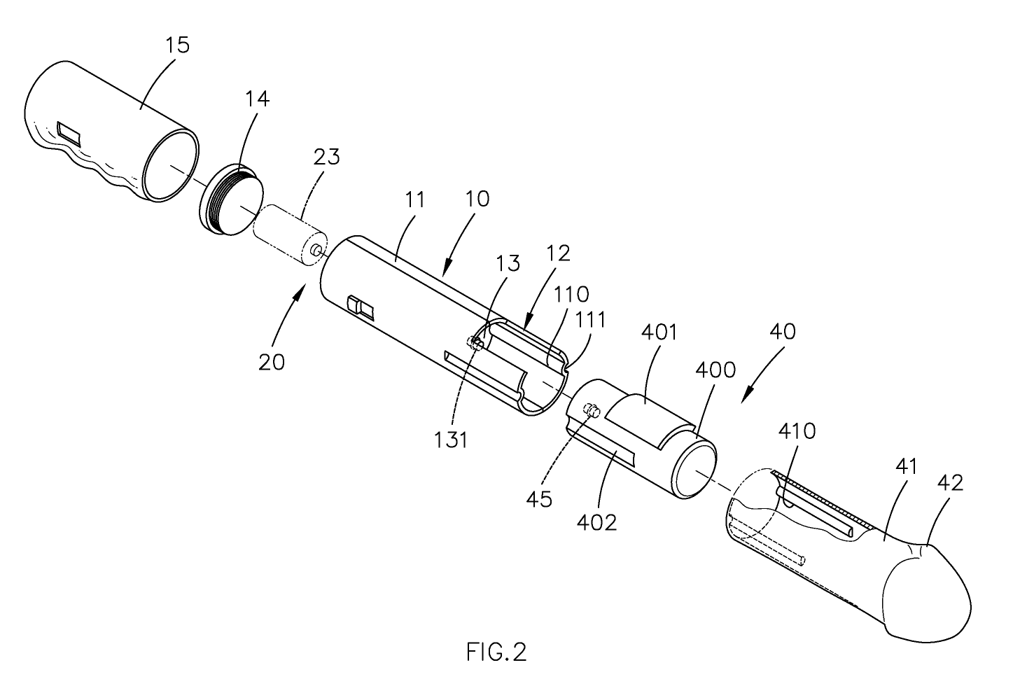

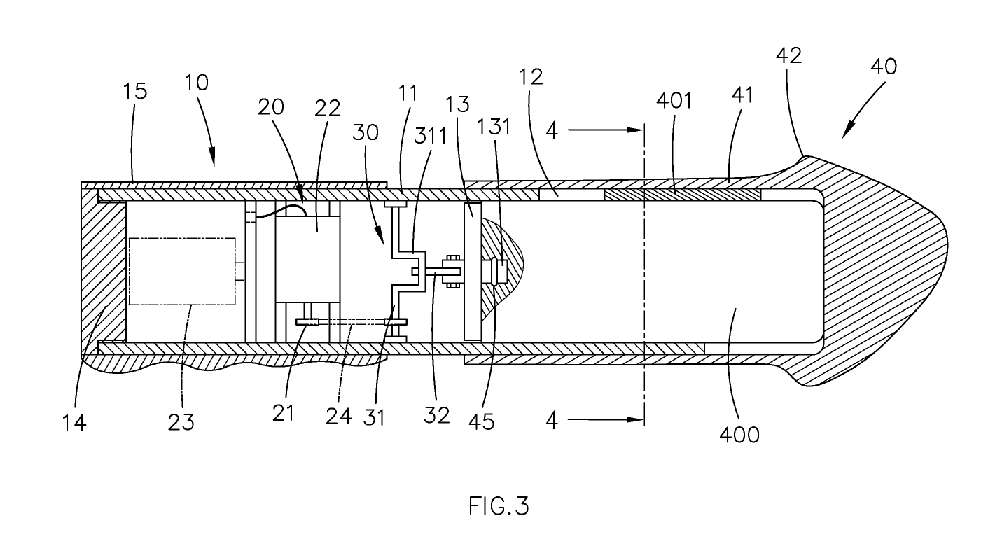

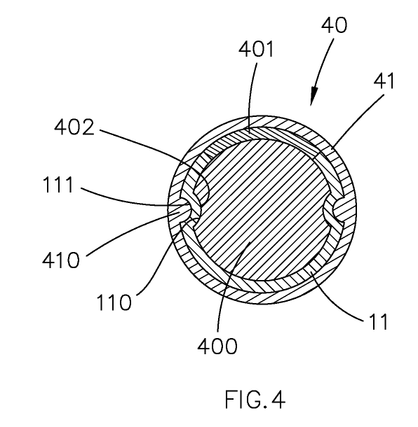

Referring to the drawings and initially to FIGS. 1-4, a massaging device for a sexual purpose in accordance with the preferred embodiment of the present invention comprises a main body 10, a power source 20, a reciprocating mechanism 30, and a sliding unit 40.

The main body 10 includes a housing 11 having a first end formed with an opening 12, a slide 13 slidably mounted in the housing 11, an end cap 14 mounted on a second end of the housing 11, and a grip portion 15 mounted on an outside of the housing 11. The grip portion 15 of the main body 10 is made of a soft anti-skid material to facilitate a user holding the grip portion 15 of the main body 10. The opening 12 is formed in a side of the housing 11 of the main body 10. The housing 11 of the main body 10 has an inner wall formed with at least one inwardly and axially extending guide rail 110 and an outer wall formed with at least one axially extending slideway 111.

The power source 20 is mounted in the housing 11 of the main body 10 and located between the slide 13 and the end cap 14. The power source 20 includes an output shaft 21 rotatably mounted in the housing 11 of the main body 10, a motor (or an electromagnetic valve) 22 connected to the output shaft 21 to rotate the output shaft 21, a battery set 23 connected to the motor 22 to supply an electric power to the motor 22, and a follower 24 mounted on and driven by the output shaft 21. Preferably, the follower 24 of the power source 20 is a chain or a gear train.

The reciprocating mechanism 30 is mounted in the housing 11 of the main body 10 and located between the slide 13 and the end cap 14. The reciprocating mechanism 30 is connected to and driven by the follower 24 of the power source 20 so that the reciprocating mechanism 30 is rotatably mounted in the housing 11 of the main body 10. The reciprocating mechanism 30 includes a rotation shaft 31 rotatably mounted in the housing 11 of the main body 10 and formed with a cycling portion 311 which is rotatable with the rotation shaft 31 to perform an eccentrically reciprocal motion, and a linking member 32 having a first end pivotally connected with and moved by the cycling portion 311 of the rotation shaft 31 and a second end pivotally connected with the slide 13 of the main body 10. The rotation shaft 31 of the reciprocating mechanism 30 is driven by the follower 24 of the power source 20. Preferably, the rotation shaft 31 of the reciprocating mechanism 30 is a crank shaft, and the cycling portion 311 of the rotation shaft 31 is a crank portion on the crank shaft.

The sliding unit 40 is movably mounted on the housing 11 of the main body 10 reciprocally and includes an elastic massaging jacket 41 movably mounted on the outside of the housing 11 of the main body 10 reciprocally, and a drive member 400 movably mounted in the housing 11 of the main body 10 reciprocally and connected to the elastic massaging jacket 41 to move the elastic massaging jacket 41 reciprocally. The drive member 400 of the sliding unit 40 has a first end extended into the housing 11 of the main body 10 and connected to and moved by the slide 13 of the main body 10 so that the drive member 400 of the sliding unit 40 is moved reciprocally by the linking member 32 of the reciprocating mechanism 30. The drive member 400 of the sliding unit 40 has a second end located adjacent to the elastic massaging jacket 41. The drive member 400 of the sliding unit 40 has a periphery formed with at least one guide groove 402 slidably mounted on the guide rail 110 of the housing 11. The drive member 400 of the sliding unit 40 is provided with a connecting portion 401 protruded from the opening 12 of the housing 11 and connected to the elastic massaging jacket 41. The elastic massaging jacket 41 of the sliding unit 40 has an inner wall formed with at least one slide rail 410 slidably mounted in the slideway 111 of the housing 11. The elastic massaging jacket 41 of the sliding unit 40 has a distal end formed with a massaging portion 42. Preferably, the massaging portion 42 of the elastic massaging jacket 41 has a shape of a human sexual organ, such as the male penis.

In operation, when the output shaft 21 of the power source 20 is rotated, the follower 24 is driven by the output shaft 21 to rotate the rotation shaft 31 of the reciprocating mechanism 30, so that the cycling portion 311 of the rotation shaft 31 is rotated eccentrically about an axis of the rotation shaft 31 to move the linking member 32 which moves the slide 13 which moves the drive member 400 which moves the elastic massaging jacket 41 which moves the massaging portion 42. Thus, the elastic massaging jacket 41 is movable on the housing 11 of the main body 10 linearly and reciprocally, and the massaging portion 42 of the elastic massaging jacket 41 is movable to massage a female sexual organ, such as the female vagina, so as to satisfy the sexual requirement.

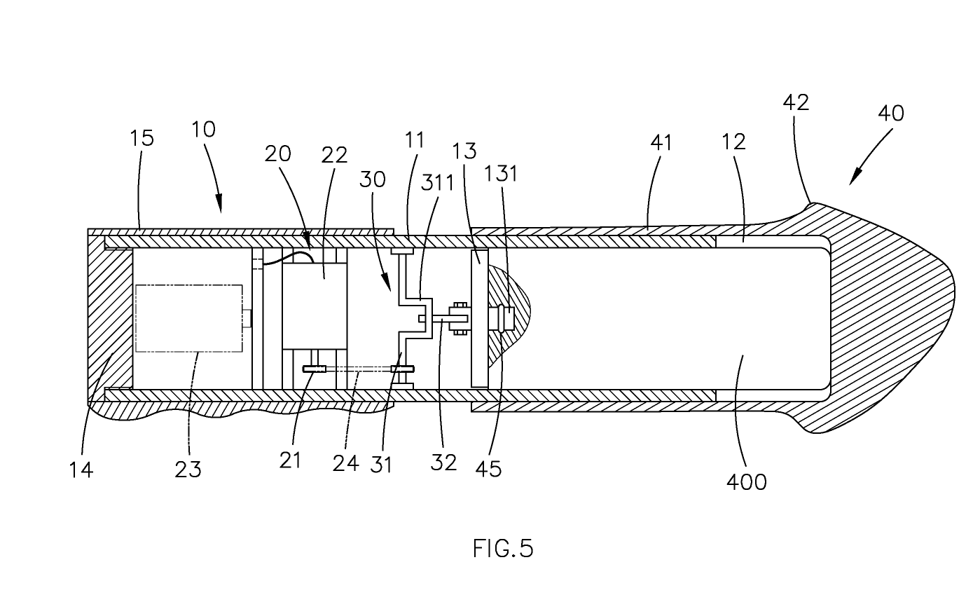

Referring to FIG. 5, the opening 12 is formed in a top of the first end of the housing 11 of the main body 10, and the connecting portion 401 of the drive member 400 is undefined. In addition, the drive member 400 of the sliding unit 40 has a second end connected to the elastic massaging jacket 41 to move the elastic massaging jacket 41 reciprocally.

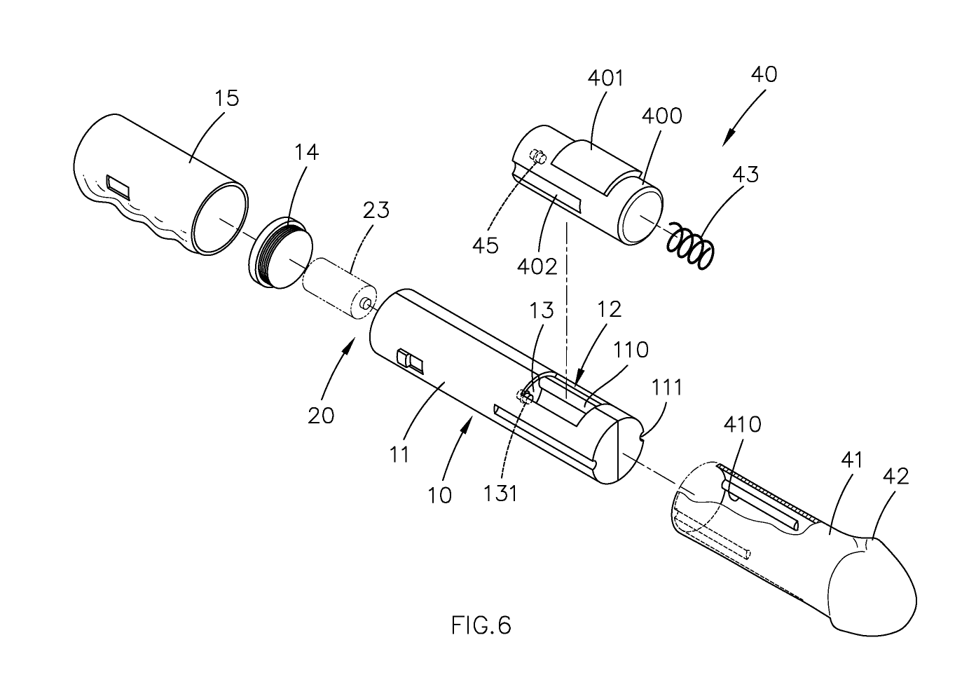

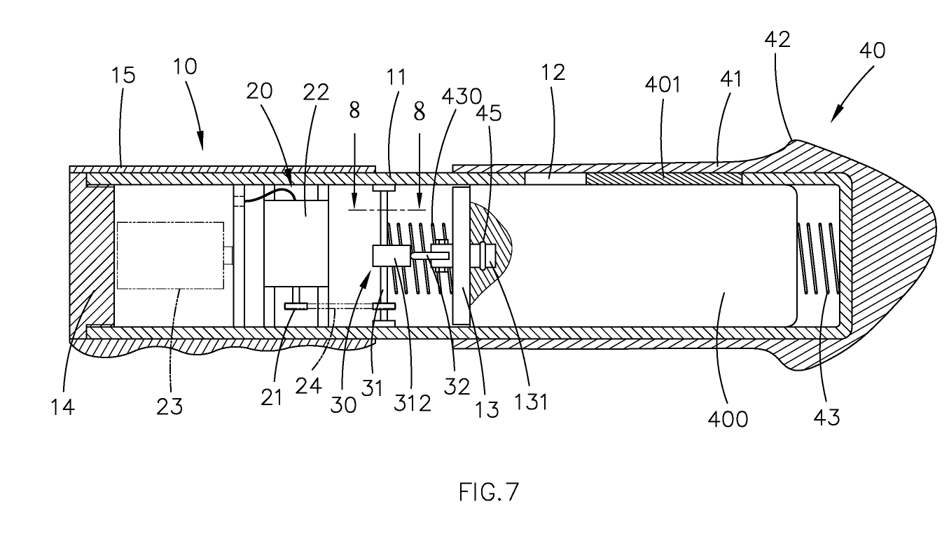

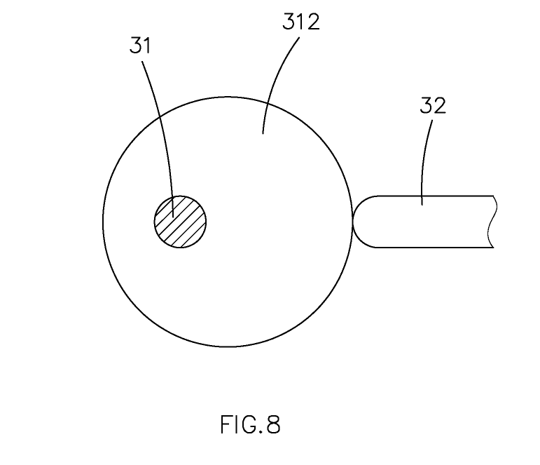

Referring to FIGS. 6-8, the rotation shaft 31 of the reciprocating mechanism 30 is provided with a cam 312 which is rotatable with the rotation shaft 31 to perform an eccentrically reciprocal motion. The top of the first end of the housing 11 of the main body 10 has a closed wall, and an elastic member 43 is mounted in the housing 11 of the main body 10 and biased between the drive member 400 of the sliding unit 40 and the closed wall of the housing 11 of the main body 10. In addition, a secondary elastic member 430 is mounted in the housing 11 of the main body 10 and biased between the slide 13 of the main body 10 and the rotation shaft 31 of the reciprocating mechanism 30.

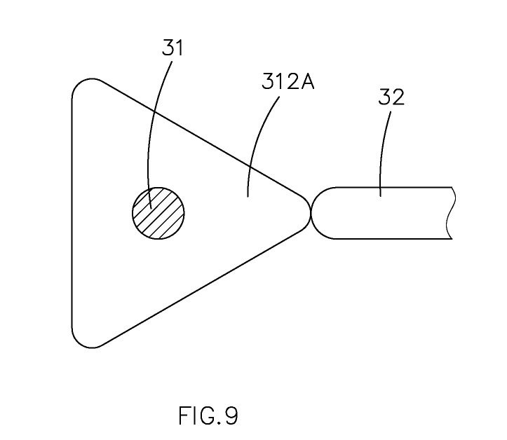

Referring to FIG. 9, the rotation shaft 31 of the reciprocating mechanism 30 is provided with a triangular portion 312A which is rotatable with the rotation shaft 31 to perform an eccentrically reciprocal motion.

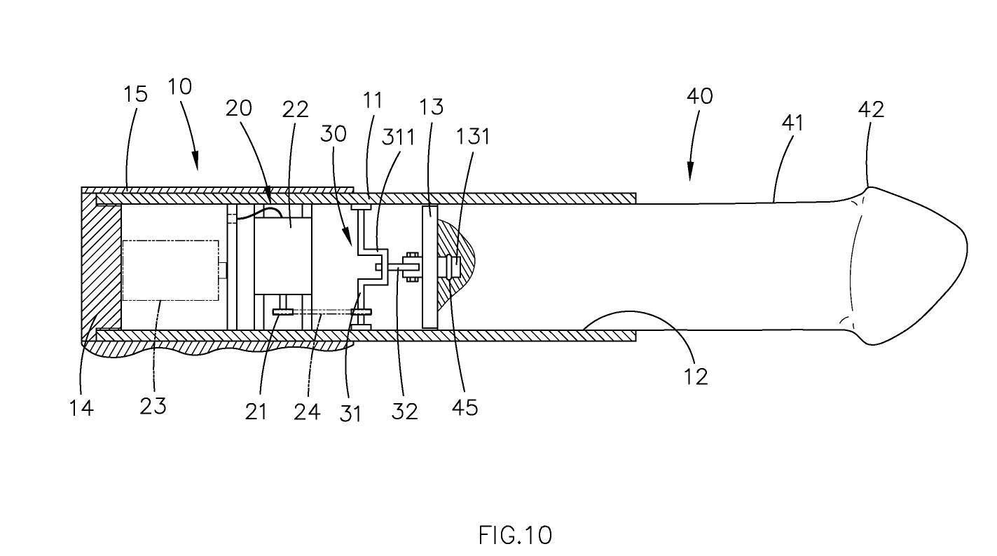

Referring to FIG. 10, the drive member 400 of the sliding unit 40 is undefined, and the sliding unit 40 includes an elastic massaging jacket 41 movably mounted in the housing 11 of the main body 10 reciprocally. The elastic massaging jacket 41 of the sliding unit 40 has a first end connected to and moved by the slide 13 of the main body 10 so that the elastic massaging jacket 41 of the sliding unit 40 is moved reciprocally by the linking member 32 of the reciprocating mechanism 30. The elastic massaging jacket 41 of the sliding unit 40 has a second end protruded from the opening 12 of the housing 11 and formed with a massaging portion 42.

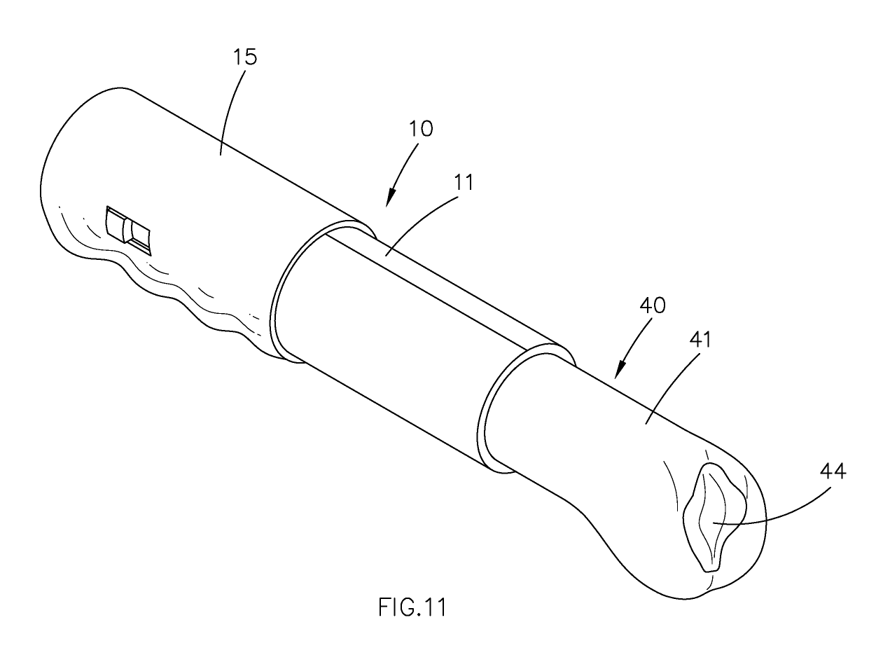

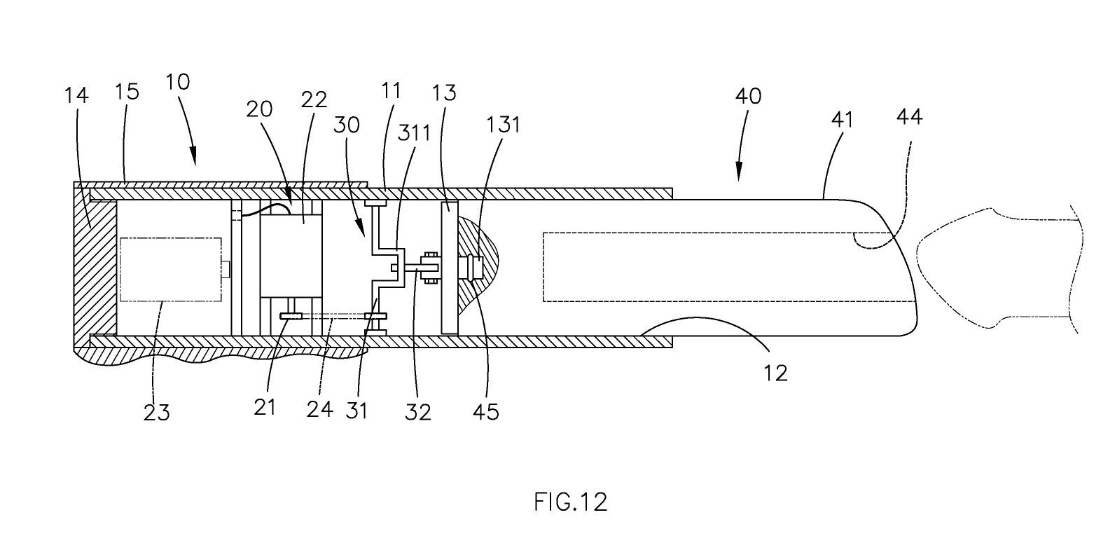

Referring to FIGS. 11 and 12, the elastic massaging jacket 41 of the sliding unit 40 has a second end protruded from the opening 12 of the housing 11 and formed with a central channel 44. Preferably, the central channel 44 of the elastic massaging jacket 41 has a shape of a human sexual organ, such as the female vagina. Thus, the central channel 44 of the elastic massaging jacket 41 is movable to massage a male sexual organ, such as the male penis, so as to satisfy the sexual requirement.

In the preferred embodiment of the present invention, the sliding unit 40 is formed with a locking hole 45, and the slide 13 of the main body 10 is formed with a locking post 131 inserted into and locked in the locking hole 45 to attach the sliding unit 40 to the slide 13 of the main body 10, so that the sliding unit 40 is movable with the slide 13 of the main body 10.

Accordingly, the sliding unit 40 is movable on the housing 11 of the main body 10 linearly and reciprocally to massage a human sexual organ automatically and to provide a comfortable sensation to the human sexual organ so as to satisfy the sexual requirement.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

What is claimed is:

1. A massaging device, comprising:
a main body;
a sliding unit movably mounted on the main body linearly and reciprocally;
a reciprocating mechanism mounted in the main body and connected to the sliding unit to drive the sliding unit reciprocally;
a power source mounted in the main body and connected to the reciprocating mechanism to drive the reciprocating mechanism.

2. The massaging device in accordance with claim 1, wherein the main body includes a housing.

3. The massaging device in accordance with claim 2, wherein the power source is mounted in the housing of the main body and includes an output shaft rotatably mounted in the housing of the main body, and a follower mounted on and driven by the output shaft.

4. The massaging device in accordance with claim 3, wherein the power source further includes a motor connected to the output shaft to rotate the output shaft, and a battery set connected to the motor to supply an electric power to the motor.

5. The massaging device in accordance with claim 3, wherein the reciprocating mechanism is connected to and driven by the follower of the power source so that the reciprocating mechanism is rotatably mounted in the housing of the main body.

6. The massaging device in accordance with claim 5, wherein the reciprocating mechanism is mounted in the housing of the main body and includes a rotation shaft rotatably mounted in the housing of the main body and driven by the follower of the power source.

7. The massaging device in accordance with claim 6, wherein the rotation shaft of the reciprocating mechanism is formed with a cycling portion which is rotatable with the rotation shaft to perform an eccentrically reciprocal motion, the reciprocating mechanism further includes a linking member pivotally connected between the rotation shaft and the sliding unit, and the linking member has a first end pivotally connected with and moved by the cycling portion of the rotation shaft and a second end pivotally connected with the sliding unit.

8. The massaging device in accordance with claim 7, wherein the rotation shaft of the reciprocating mechanism is a crank shaft, and the cycling portion of the rotation shaft is a crank portion on the crank shaft.

9. The massaging device in accordance with claim 6, wherein the sliding unit includes an elastic massaging jacket movably mounted on an outside of the housing of the main body reciprocally, and a drive member movably mounted in the housing of the main body reciprocally and connected to the elastic massaging jacket to move the elastic massaging jacket reciprocally.

10. The massaging device in accordance with claim 9, wherein the drive member of the sliding unit has a first end extended into the housing of the main body and connected to and moved by the reciprocating mechanism.

11. The massaging device in accordance with claim 9, wherein the drive member of the sliding unit is provided with a connecting portion protruded from an opening of the housing and connected to the elastic massaging jacket.

12. The massaging device in accordance with claim 9, wherein the elastic massaging jacket of the sliding unit has a distal end formed with a massaging portion.

13. The massaging device in accordance with claim 10, wherein the drive member of the sliding unit has a second end connected to the elastic massaging jacket to move the elastic massaging jacket reciprocally.

14. The massaging device in accordance with claim 9, wherein the rotation shaft of the reciprocating mechanism is provided with a cam which is rotatable with the rotation shaft to perform an eccentrically reciprocal motion.

15. The massaging device in accordance with claim 14, wherein the housing of the main body has an end having a closed wall located between the drive member and the elastic massaging jacket of the sliding unit, and the massaging device further comprises an elastic member mounted in the housing of the main body and biased between the drive member of the sliding unit and the closed wall of the housing of the main body.

16. The massaging device in accordance with claim 9, wherein the rotation shaft of the reciprocating mechanism is provided with a triangular portion which is rotatable with the rotation shaft to perform an eccentrically reciprocal motion.

17. The massaging device in accordance with claim 6, wherein the sliding unit includes an elastic massaging jacket movably mounted in the housing of the main body reciprocally and has a first end connected to and moved by the reciprocating mechanism.

18. The massaging device in accordance with claim 17, wherein the elastic massaging jacket of the sliding unit has a second end protruded from an opening of the housing and formed with a massaging portion or a central channel.

19. The massaging device in accordance with claim 2, wherein the main body further includes a slide slidably mounted in the housing and driven by the reciprocating mechanism, and the sliding unit is movable with the slide of the main body.

20. The massaging device in accordance with claim 19, wherein the sliding unit is formed with a locking hole, and the slide of the main body is formed with a locking post inserted into and locked in the locking hole to attach the sliding unit to the slide of the main body, so that the sliding unit is movable with the slide of the main body.
